(19) United States
(12) Patent Application Publication
(10) Pub. No.: US 2003/0215531 A1
(43) Pub. Date: Nov. 20, 2003

(54) Compositions and methods of use for extracts of Rutaceae plants

(76) Inventors: Martin Stogniew, Blue Bell, PA (US); Walter Chambliss, Memphis, TN (US)

Correspondence Address: William J. Brucker, Esq., STETINA BRUNDA GARRED & BRUCKER, Suite 250, 75 Enterprise, Aliso Viejo, CA, 92656, US

(21) Appl. No.: 10/394,308

(22) Filed: Mar. 20, 2003

Related U.S. Application Data
(63) Continuation of application No. 09/948,019, filed on Sep. 6, 2001, now abandoned.
(60) Provisional application No. 60/235,920, filed on Sep. 28, 2000.

(51) Int. Cl. A61K 35/78

(52) U.S. Cl. 424/730; 424/725

(57) ABSTRACT

The invention relates to compositions and methods for preventing, treating, or managing anxiety, pain, chronic pain, depression, and disorders such as premenstrual syndrome comprising the administration of a prophylactically and therapeutically effective amount of Rutaceae plant or extracts thereof to a mammal in need of such therapy. In a preferred embodiment, the mammal is human and the extracts are substantially free of the compounds obacunone or limonin. The invention also relates to compositions and methods for preventing, treating, or managing separation anxiety in domestic animals comprising the administration of a prophylactically and therapeutically effective amount of Rutaceae plant or extracts thereof to an animal in need of such therapy.

This application claims the benefit of U.S. Provisional Application No. 60/235,920 filed Sep. 28, 2000, which is incorporated herein by reference.

### 1. INTRODUCTION

The present invention relates to novel methods and compositions for the treatment, prevention, or management of anxiety, pain, chronic pain, depression, disorders such as premenstrual syndrome, weight loss, cold, appetite loss, sleeplessness, and fatigue. The methods and compositions utilize plants, portions thereof or extracts therefrom belonging to the Rutaceae family, preferably the extract is substantially free of the compounds obacunone or limonin. In addition, the methods and compositions utilize mixtures of specific small molecules extracted from the Rutaceae plant, such as, but not limited to, berberine, phellodendrine, and magnoflorine. The unique compositions of the invention may also comprise various amounts of the Rutaceae plant, plant extract, plant extracts combined with different amounts of biologically active small molecules or other therapeutic agents. These compositions are particularly useful for the treatment of anxiety, pain, chronic pain, depression, premenstrual syndrome, weight loss, cold, appetite loss, sleeplessness and fatigue in humans. The invention also encompasses various modes of administration of the therapeutic extracts or other compositions of the invention. Finally, the compositions of the invention can be formulated for veterinary use since they are also useful for the treatment of separation anxiety in domestic animals.

### 2. BACKGROUND OF THE INVENTION

The recent growth in sales of natural products labeled as dietary supplements in the United States has renewed scientific interest in the study of the prophylactic and therapeutic effects of multi-component botanical products. Unlike single entity pharmaceutical products, botanical products comprise a large number of diverse chemical constituents that often act synergistically to exert a desired biological effect. The type of extraction process utilized and the manner in which the formulation is standardized have dramatic effects on the pharmacological activity of the final product. The development of new botanical products requires multidisciplinary effort consisting of expertise in ethnobotany, natural product chemistry, analytical chemistry, pharmacology, and natural product extraction.

### 2.1 Anxiety

All humans experience fear and anxiety. Fear is an emotional, physiologic and behavioral response to a recognized external threat. Anxiety is an unpleasant emotional state, often accompanied by physiologic changes and behaviors similar to those caused by fear. Adaptive anxiety helps people prepare, practice, and rehearse so that their functioning is improved and helps them be appropriately cautious in potentially dangerous situations. Maladaptive anxiety causes distress and dysfunction. As anxiety increases, performance efficiency increases proportionately but only to an optimal level, beyond which performance efficiency decreases with further increase in anxiety.

Anxiety disorders are more common than any other class of psychiatric disorder. However, they are often not recognized and consequently not treated.

The causes of anxiety disorders are not fully known, but both physiological and psychological factors are involved. Physiologically, all thoughts and feelings may be understood as resulting from electrochemical processes in the brain, but this fact tells little about the complex interactions among the >200 neurotransmitters and neuromodulators of the brain and about normal vs. abnormal arousal and anxiety. Psychologically, anxiety is viewed as a response to environmental stressors such as exposure to a life-threatening disaster.

Anxiety can arise suddenly, as in panic, or gradually over many minutes, hours or even days. Anxiety may last from a few seconds to years; longer duration is often associated with anxiety disorders. Anxiety ranges in intensity, from barely noticeable qualms to complete panic, it's most extreme form. One person's passion may be another's anxiety and the ability to tolerate anxiety varies from person to person. Deciding when anxiety is so severe that it is a disorder depends on several variables, and physicians differ in making the diagnosis. If anxiety is very distressing, interferes with functioning, and does not stop spontaneously within a few days, an anxiety disorder is present and merits treatment.

Diagnosis of a specific anxiety disorder is based largely on its characteristic symptoms and signs. A family history of anxiety disorders is helpful, because many patients appear to have inherited a predisposition to the same anxiety disorders their relatives have as well as a general susceptibility to other anxiety disorders.

### 2.2 Depression

Depression is a feeling of intense sadness; it may follow a recent loss or other sad event but is out of proportion to that event and persists beyond an appropriate length of time.

After anxiety, depression is the most common psychiatric disorder. An estimated 10 percent of the people who see their doctors for what they think is a physical problem are actually experiencing depression. Depression typically begins in the 20s, 30s, or 40s. People born in the latter part of the 20^{th} century seem to have higher rates of depression than those of previous generations, in part because of higher rates of substance abuse.

An episode of depression typically lasts for 6 to 9 months, but in 15 to 20 percent of the people, it lasts for 2 years or more. Episodes generally tend to recur several times over a lifetime.

The causes of depression are not fully understood. A number of factors may make a person more likely to experience depression, such as a family tendency (heredity), side effects of certain medications, an introverted personality, and emotionally upsetting events, particularly those involving a loss. Depression may also arise or worsen without any apparent or significant life stress.

Women are twice as likely as men to experience depression, though the reasons are not entirely clear. Psychologic studies show that women tend to respond to adversity by withdrawing into themselves and blaming themselves. Of biologic factors, hormones are the ones most involved. Changes in hormone levels, which can create mood changes shortly before menstruation (premenstrual tension) and after childbirth (postpartum depression), might play some role in women. Abnormal thyroid function, which is fairly common in women, may also be a factor.

Depression that follows a traumatic event, such as the death of a loved one, is called situational depression. Some people become temporarily depressed in reaction to certain holidays or meaningful anniversaries. Depression with an apparent precipitating event is called endogenous depression. These distinctions, however, are not very important, since the effects and treatment of the depression are similar.

Depression may also occur with, or be caused by, a number of physical diseases or disorders. Physical disorders may cause a depression directly (such as when thyroid disease affects hormone levels, which can induce depression) or indirectly (such as when rheumatoid arthritis causes pain and disability, which can lead to depression). Often, depression that results from a physical disorder has both direct and indirect causes.

Several types of drugs, tricyclic antidepressants, selective serotonin reuptake inhibitors, monoamine oxidates inhibitors, and psychostimulants are available, but they must be taken regularly for at least several weeks before they begin to work. The chances that any given antidepressant will work for a particular person are about 65 percent. The adverse effects vary with each type of drug. The tricyclic antidepressants often cause sedation and lead to weight gain. They can also be associated with an increased heart rate, a decrease in blood pressure when the person stands, blurred vision, dry mouth, confusion, constipation, difficulty in starting to urinate, and delayed orgasm. These effects are called anticholinergic effects and are often more pronounced in the elderly.

The selective serotonin reuptake inhibitors (SSRIs) represent a major improvement in the treatment of depression in that they tend to cause fewer adverse effects than they tricyclic antidepressants. Also they are generally quite safe in people who have depression and a coexisting physical disorder. Although they can cause nausea, diarrhea, and headache, these adverse effects are either mild or go away with continued use. For these reasons, doctors often select SSRIs first when treating depression. SSRIs are particularly useful in the treatment of dysthymia, which requires long-term drug therapy. Moreover, SSRIs are quite effective in obsessive-compulsive disorder, panic disorder, social phobia, and bulimia, which often coexist with depression.

### 2.3 Pain

Pain is an unpleasant sensation signaling that the body is damaged or threatened with an injury. Pain begins at special pain receptors scattered throughout the body. These pain receptors transmit messages as electrical impulses along nerves to the spinal cord and then upward to the brain. Sometimes the signal evokes a reflex response when it reaches the spinal cord; when this happens, a signal is immediately sent back along motor nerves to the original site of the pain, triggering the muscles to contract. An example of a reflex response is the immediate pull-away reaction upon inadvertently touching something very hot. The pain signal is also relayed to the brain. Only when the brain processes the signal and interprets it as pain does a person become consciously aware of it.

Pain receptors and their nerve pathways differ in different parts of the body. Because of this, pain sensation varies with the type and location of injury. For example, pain receptors in the skin are plentiful and capable of transmitting precise information, such as where an injury is located and whether the cause was sharp as a knife wound or dull such as pressure, heat, or cold. In contrast, pain signals from the intestine are limited and imprecise. The intestine can be pinched, cut, or burned without generating a pain signal. However, stretching and pressure can cause severe intestinal pain, even from something as relatively harmless as a trapped gas bubble. The brain cannot identify the precise source of intestinal pain; rather, the pain is difficult to locate and is likely to be felt over a large area.

Pain felt in some areas of the body may not accurately represent where the trouble is, because pain can be referred to another area. Referred pain happens because signals from several areas of the body often lead into the same nerve pathways going to the spinal cord an brain. For example, pain from a heart attack may be felt in the neck, jaws, arms, or abdomen, and pain from a gallbladder attack may be felt in the shoulder.

People differ remarkably in their ability to tolerate pain. One person may find the pain of a small cut or bruise intolerable, while another person can tolerate a major accident or knife wound with little complaint. Ability to withstand pain varies according to mood, personality, and circumstance. In a moment of excitement during an athletic match, an athlete may not notice a severe bruise but will likely be very aware of the pain after the match, particularly if the team lost.

Pain may even change with age. As people age, the complain less of pain, perhaps because changes in the body decrease the sensation of pain. On the other hand, the elderly may simply be more stoic than younger people. Pain may be sharp of dull, intermittent or constant, throbbing or consistent, at a single site or all over. The intensity can vary from minor to intolerable. No laboratory test can prove the presence or severity of pain.

### 2.4 Chronic Pain

Pain is a common symptom, reflecting either physical (i.e., the result of tissue injury or inflammation) or emotional discomfort. Pain is a complex subjective phenomenon comprised of a sensation reflecting real or potential tissue damage, and the affective response this generates. Pain may be classified as either acute or chronic, and it is of a variety of particular types. Acute pain is an essential biologic signal of the potential for, or the extent of, tissue injury. In contrast, chronic pain is physically and psychologically debilitating, and it no longer serves its adaptive biologic role. Chronic pain is usually defined broadly and arbitrarily as pain persisting >1 month beyond the resolution of an acute tissue injury, pain persisting or recurring for >3 months, or pain associated with tissue injury that is expected to continue or progress. In many patients, organic disease may be insufficient to explain the degree of pain. Chronic pain may be associated with conditions including but not limited to osteoarthritis, rheumatoid arthritis, soft tissue pain syndromes, and headaches. Vegetative signs, e.g., lassitude, sleep disturbance, decreased appetite, loss of taste for food, weight loss, diminished libido, constipation, often develop gradually and depression may follow.

### 2.5 Premenstrual Syndrome

Premenstrual syndrome (PMS, premenstrual dysphoric disorder, late luteal phase dysphoric disorder) is a condition in which a variety of symptoms, including nervousness irritability, emotional upset, depression; headaches, tissue swelling, and breast tenderness, may occur during the 7 to 14 days before a menstrual period begins.

Premenstrual syndrome may be related to the fluctuations in estrogen and progesterone levels that occur during the menstrual cycle. Estrogen causes fluid retention, which probably explains the weight gain, tissue swelling, breast tenderness, and bloating. Other hormonal and metabolic changes may also be involved.

The type and intensity of symptoms vary from woman to woman and from month to month in the same woman. The broad range of physical and psychologic symptoms can temporarily upset a woman's life. Women who have epilepsy may have more seizures than usual. Woman who have a connective tissue disease, such as systemic lupus erythematosus, or rheumatoid arthritis, may have flare-ups at this time.

Usually, symptoms occur a week or two before the menstrual period, and stop when the next period begins. Women close to menopause may have symptoms that persist through and after the menstrual period. The symptoms of premenstrual syndrome are often followed each month by a painful period.

Taking a combination oral contraceptives, which comprise estrogen and progestin, helps reduce the fluctuations in estrogen and progesterone levels. Fluid retention and bloating are often relieved by reducing the intake of salt and taking a mild diuretic, such as spironolactone, just before symptoms are expected to begin. Other dietary changes, such as decreasing the amount of sugar, caffeine, and alcohol consumed; eating more carbohydrates; and having more frequent meals may help. Dietary supplements comprising calcium and magnesium may be beneficial. Taking vitamin B supplements, especially B₆ (pyridoxine), may reduce some symptoms, although the benefits of vitamin B₆ have recently been questioned and a dose that is too high may be harmful. Nonsteroidal anti-inflammatory drugs may help relieve headaches, pain from uterine cramps, and joint aches.

### 2.6 Rutaceae Plant Extracts

Extracts from plants belonging to the family Rutaceae have been and may still be used in Chinese herbalism. The amur cork tree, Phellodendron amurense, called Huang Bai in China, is considered to be one of the 50 fundamental herbs in Chinese herbalism and one that should be used with care. (J. A. Duke and E. S. Ayensu, Medicinal Plants of China, Reference Publications, Inc. 1985; A. Chevallier, The Encyclopedia of Medicinal Plants, Dorling Kindersley, London 1996). The bark acts strongly on the kidneys and is regarded as a detoxicant for hot damp conditions. (D. Brown, Encyclopedia of Herbs and their Uses, Dorling Kindersley, London 1995). Research has shown that the plant is useful in the treatment of meningitis and conjunctivitis. (The Encyclopedia of Medicinal Plants).

The bark of Phellodendron amurense, is reportedly used as an antibacterial, antirheumatic, aphrodisiac, bitter stomachic, cholagogue, diuretic, expectorant, febrifuge, hypoglycemia, ophthalmic, skin vasodilator, tonic, and central nervous stimulant (CNS). (Him-Che Yeung, Handbook of Chinese Herbs and Formulas, Institute of Chinese Medicine, Los Angeles 1985; Rev. G. A. Stuart, Chinese Materia Medica, Tapei southern Materials Centre; U.S. Pat. No. 5,344,648). It is taken internally in the treatment of acute diarrhea, dysentery, jaundice, vaginal infections, including trichomonas, acute urinary tract infections, enteritis, bolis, abscesses, night sweats, and skin diseases. (The Encyclopedia of Medicinal Plants; Encyclopedia of Herbs and their Uses). The bark of 10 year old trees is harvested in the winter or spring and dried for later use. Id.

To date, there are a number of disorders for which there is no dietary supplement available to either prevent or alleviate the disorder or symptoms associated therewith. It is desirable to discover and develop dietary supplements or pharmaceutical compositions based upon natural materials that are both safe and effective. It is particularly desirable to develop plant extracts for the prevention, treatment, or control of anxiety, depression, pain, chronic pain, premenstrual syndrome, and symptoms discussed above.

### 3. SUMMARY OF THE INVENTION

The invention described herein encompasses compositions and methods of treating or preventing anxiety, depression, pain, chronic pain, and disorders such as premenstrual syndrome. The methods comprise the administration of a therapeutically or prophylactically effective amount of an extract from the Rutaceae family, particularly to a human in need of such therapy. The plants belonging to the Rutaceae family include plants within the genus Phellodendron, Citrus, Evodia, or Dictamnus. The extracts of the invention are prepared using solvents such as lower alcohols, water, and mixtures thereof.

Preferably the compositions comprise an aqueous Rutaceae plant extract substantially free of obacunone and limonin. Alternatively, the compositions comprise Rutaceae plant extracts soluble in a lower alcohol, water, and mixtures thereof, or at least two compounds selected from the group consisting of phellodendrine, magnoflorine and berberine. The compositions may contain a pharmaceutically acceptable carrier, excipient, or diluent. In one embodiment, the compositions are pharmaceutical compositions for human use or veterinary compositions for use with domestic animals. The pharmaceutical compositions can be included as unit dosage suitable for parenteral, oral, or intravenous administration to a human. Alternatively, the compositions are dietary supplements or food compositions suitable for human or animal consumption.

The Rutaceae plant extract is obtained by cutting or pulverizing a plant of the family Rutaceae, extracting the cut or powdered plant parts with a suitable aqueous solvent for a time sufficient to form an extract. Subsequently, the extract is concentrated under reduced pressure and optionally dried. Further, the extract may optionally be purified to remove undesirable components.

The compositions of the invention typically comprise about 25% to about 100% of the Rutaceae plant extract by weight. The compositions may include additional ingredients such at least one anxiolytic agent present in an amount of about 0.5% to about 10%, at least one anti-depressant agent present in an amount of about 0.5% to about 10%, at least one analgesic present in the amount of about 5% to about 80%, an agent for treating premenstrual syndrome present in an amount of about 5% to about 80%, or an anti-inflammatory agent present in an amount of about 5% to about 80% by weight of the composition.

The methods described herein comprise methods for treating, preventing, and managing anxiety, depression, pain, chronic pain, or premenstrual syndrome by administering a therapeutically effective amount of a composition comprising a Rutaceae plant or plant extract wherein the Rutaceae plant belongs to the genus Phellodendron, Citrus, Evodia, or Dictamnus. The method of treating the above mentioned conditions includes administering an extract obtained using a solvent selected from the group consisting of a lower alcohol, water, and mixtures thereof. Alternatively, the extract can be an aqueous extract substantially free of obacunone or limonin or comprising at least two compounds selected from the group consisting of phellodendrine, magnoflorine, and berberine.

In another embodiment of the invention, a method for the treatment of separation anxiety in domestic animal comprises administering to an animal in need of treatment a therapeutically effective amount of a Rutaceae plant extract wherein the Rutaceae plant belongs to the genus Phellodendron, Citrus, Evodia, or Dictamnus. The method for treating separation anxiety in domestic animals comprises administering a Rutaceae plant extract obtained using a solvent selected from the group consisting of a lower alcohol, water, and mixtures thereof. Alternatively, the extract can be an aqueous extract substantially free of obacunone or limonin or comprising at least two compounds selected from the group consisting of phellodendrine, magnoflorine, and berberine.

In yet another embodiment of the invention, the methods described herein comprise methods for treating, preventing, and managing anxiety, depression, and premenstrual syndrome by administering to a mammal in need thereof a therapeutically effective amount of phellodendrine, magnoflorine, or berberine, pharmaceutically acceptable salts thereof, or compositions thereof. Compositions having phellodendrine and magnoflorine can also be used for treating, preventing, and managing pain and chronic pain by administering to a mammal in need thereof a therapeutically effective amount.

### 3.1 Definitions

As used herein, unless otherwise specified, the term “Rutaceae plant” includes, but is not limited to, any part of a plant within the family Rutaceae. The plant parts may include plant bodies preferably the stalk, leaves, fruit or rind, bark, flowers, stems, roots, or the seeds. Preferred plants within the Rutaceae family are discussed below.

As used herein, a composition that is “substantially free” of a compound means that the composition contains less than about 15% by weight, preferably less than about 3% by weight, more preferably less than about 2% by weight, and most preferably less than about 1% by weight of the compound.

As used herein, unless otherwise specified, the term “treating anxiety” or “treatment of anxiety” includes, but is not limited to, preventing or reducing any unpleasant emotional state often accompanied by physiologic changes and behaviors similar to those caused by fear.

As used herein, unless otherwise specified, the term “treating depression” or “treatment of depression” means preventing or reducing the feeling of intense sadness which is disproportionate to a triggering event or persistent for a period of time beyond an appropriate length of time.

As used herein, unless otherwise specified, the term “treating premenstrual syndrome” or “treatment of premenstrual syndrome” means preventing or reducing any of the variety of symptoms, including, but not limited to, nervousness, irritability, emotional upset, depression, headaches, tissue swelling and breast tenderness, which may occur during the 7 to 14 days before a menstrual period beings.

As used herein, unless otherwise specified, the term “physiologically acceptable carrier,” includes, but is not limited to, a carrier medium that does not interfere with the effectiveness of the biological activity of the active ingredient, is chemically inert, and is not toxic to the consumer or patient to whom it is administered.

As used herein, unless otherwise specified, the term “pharmaceutically acceptable salts” refers to salts prepared from pharmaceutically acceptable non-toxic acids and bases, including inorganic and organic acids and bases.

As used herein, unless otherwise specified, the term “preventing,” includes, but is not limited to, inhibition or the averting of symptoms associated with a particular disease. As used herein, unless otherwise specified, the term “treating” refers to the administration of the composition after the onset of symptoms of the disease or disorder whereas “preventing” refers to the administration prior to the onset of the symptoms, particularly to patients at risk of the disease or disorder.

As used herein, unless otherwise specified, the term “lower alcohol” includes, but is not limited to, straight chained or branched, substituted or unsubstituted hydrocarbon compounds having at least one hydroxyl group having one to five carbon atoms. Lower alcohols include, but are not limited to, methanol, ethanol, n-propanol, isopropanol, butanols, and mixtures thereof.

As used herein, unless otherwise specified, the term “domestic animals” includes, but is not limited to, dogs, cats, horses, cattle, pigs, sheep, hamsters, ferrets, rabbits, lizards, snakes, birds, and the like.

### 4. BRIEF DESCRIPTION OF THE FIGURES

FIG. 1 illustrates the effects of a composition of the invention on distress vocalizations (“DVoc”) and the composite pain score (“CPS”) in a study using chicks.

FIG. 2 illustrates a summary of the results of a composition of the invention on DVoc and on CPS also in chicks.

FIG. 3 illustrates a comparison of the mean CPS values for a vehicle control, Naproxen, and a composition of the invention. FIG. 3 illustrates the effect of the vehicle, Naproxen, and a composition of the invention at 100 mg/kg in five minute intervals on formalin screening model.

FIG. 4 illustrates a comparison of the CPS values for a vehicle control, Naproxen, and a composition of the invention. FIG. 4 illustrates the effect of the vehicle, Naproxen, and a composition of the invention at 200 mg/kg in five minute intervals on formalin screening model.

### 5. DETAILED DESCRIPTION OF THE INVENTION

The invention encompasses compositions and methods for preventing, treating, and managing anxiety, pain, chronic pain, depression and disorders such as premenstrual syndrome, weight loss, cold, appetite loss, sleeplessness, and fatigue comprising the administration of a prophylactically and therapeutically effective amount of Rutaceae plant or an extract thereof to a mammal in need of such therapy. In a preferred embodiment, the mammal is human and the extracts are substantially free of obacunone or limonin. In a most preferred embodiment the extracts are aqueous or lower alcohol aqueous extracts. The invention also relates to compositions and methods for preventing and treating separation anxiety in domestic animals comprising the administration of a prophylactically and therapeutically effective amount of Rutaceae plant or extracts thereof to an animal in need of such therapy.

In one embodiment of the invention, the composition for preventing anxiety comprises Rutaceae plant or extracts thereof in an amount sufficient to prevent the onset of anxiety or anxiety related symptoms. In another embodiment of the invention, for mammals already suffering from anxiety, the invention is directed to compositions and administered dosages comprising Rutaceae plant in sufficient amount to reduce anxiety or the symptoms associated with anxiety during stress inducing stimuli. In yet another embodiment of the invention, for mammals already suffering from anxiety, the invention is directed to a method for treating anxiety by administering compositions containing Rutaceae plant extract in a therapeutically sufficient amount to treat anxiety.

In yet another embodiment of the invention, the composition for preventing depression comprises Rutaceae plant or extracts thereof in an amount sufficient to prevent the onset of depression or depression related symptoms. In yet another embodiment of the invention, the composition for treating depression is in sufficient amount and regularly administered dosage to reduce or eliminate depression or depression related symptoms in mammals suffering from depression. In yet another embodiment of the invention, for mammals already suffering from depression, the invention is directed to a method for treating depression by administering compositions containing Rutaceae plant extract in a therapeutically sufficient amount to either prevent or treat depression.

In another embodiment of the invention, the composition for preventing pain comprises Rutaceae plant or extracts thereof in an amount sufficient to prevent the onset of pain or pain related symptoms. In another embodiment of the invention, the composition for treating pain is in sufficient amount and regularly administered dosage to reduce or eliminate pain related symptoms in mammals suffering from pain. In yet another embodiment of the invention, for mammals already suffering from pain, the invention is directed to a method for treating pain by administering compositions containing Rutaceae plant extract in a therapeutically sufficient amount to either prevent or treat pain.

In another embodiment of the invention, the composition for preventing chronic pain comprises Rutaceae plant or extracts thereof in an amount sufficient to prevent the onset of chronic pain or chronic pain related symptoms. In another embodiment of the invention, the composition for treating chronic pain is in sufficient amount and regularly administered dosage to reduce or eliminate chronic pain related symptoms in mammals suffering from chronic pain. In yet another embodiment of the invention, for mammals already suffering from chronic pain, the invention is directed to a method for treating chronic pain by administering compositions containing Rutaceae plant extract in a therapeutically sufficient amount to either prevent or treat chronic pain.

In yet another embodiment of the invention, the composition for preventing premenstrual syndrome comprises Rutaceae plant or extracts thereof in an amount sufficient to prevent the onset of premenstrual syndrome and premenstrual syndrome related symptoms. In yet another embodiment of the invention, the composition for treating premenstrual syndrome is in sufficient amount and regularly administered dosage to reduce or eliminate premenstrual syndrome and premenstrual syndrome related symptoms in mammals suffering from premenstrual syndrome. In yet another embodiment of the invention, for mammals already suffering from premenstrual syndrome, the invention is directed to a method for treating premenstrual syndrome by administering compositions containing Rutaceae plant extract in a therapeutically sufficient amount to either prevent or treat premenstrual syndrome.

In a preferred embodiment of the invention, the compositions comprise a Rutaceae plant extract substantially free of obacunone or limonin. More preferably, the Rutaceae plant extract is obtained by extracting Rutaceae plant parts with a lower alcohol, water, or mixtures thereof. In a specific embodiment, the Rutaceae plant extract comprises biologically active small molecule compounds selected from the group berberine, phellodendrine, magnoflorine, and mixtures thereof.

In another preferred embodiment of the invention, the compositions comprise a Rutaceae plant or an extract thereof and an additional amount of berberine in excess to any amount that may be present in the Rutaceae plant extract. The invention encompasses compositions which !comprise berberine or a pharmaceutically acceptable salt thereof and pharmaceutically acceptable excipients, carriers, or diluents. Further, such berberine compositions can comprise anxiolytic agents, anti-depressant agents, etc. The berberine compositions comprise berberine in an amount sufficient and regularly administered dosage to prevent, treat, or manage a disorder in mammals wherein the disorder is selected from the group consisting of anxiety, depression, premenstrual syndrome, weight loss, cold, appetite loss, sleeplessness and fatigue.

The disclosure is based, in part, on the discovery that Rutaceae plant or extracts thereof, alone or in combination with other anxiolytic agents, reduces anxiety symptoms. The disclosure is also based, in part, on the discovery that Rutaceae plant or extracts thereof alone or in combination with other anti-depressants agents reduce depression. Without being limited by theory, it is believed that Rutaceae plant extracts act synergistically or at least more than additively by binding to one or more receptor sites to collectively diminish the symptoms of anxiety, pain, chronic pain, depression, premenstrual syndrome, weight loss, cold, appetite loss, sleeplessness, or fatigue without causing a sedative or addictive effect. Additionally, without being limited by theory, is it believed that the Rutaceae plant extracts of the invention, act similarly to selective serotonin reuptake inhibitors (SSRIs), such as fluoxetine (PROZAC), by altering synaptic availability of 5-hydroxytryptamine (serotonin or 5-HT) by inhibiting presynaptic reaccumulation of neuronally released serotonin.

In a preferred embodiment, the invention encompasses compositions, and the use thereof, which comprises a water-soluble extract of a Rutaceae plant. Optionally, the Rutaceae plant extract may be substantially free of any fat soluble materials such as obacunone or limonin. In a most preferred embodiment, such extracts are produced by extraction with an aqueous alcohol solvent system using such a system the extracts preferably comprise one or more, or mixtures of the following, small organic compounds which naturally occur in the plants: phellodendrine, magnoflorine, berberine and the like. It has been discovered that such extracts have a unique binding affinities despite the fact that the extracts are mixtures of components. Thus, the extracts of the invention have unique benefits in preventing, treating, or managing disorders. Moreover, the preferred extracts of the invention are useful for treating and preventing anxiety or depression without a sedative effect. Similarly, the extracts of the invention reduce or avoid adverse effects associated with certain anxiolytic and antidepressant drugs such as physical dependency, withdrawal problems, sleepiness, impaired coordination, slowed reaction time, sedation, weight gain, constipation, dry mouth, confusion, blurred vision, nausea, diarrhea or headaches.

The extracts of the invention are particularly useful for humans taking prescription medications for anxiety, depression, pain, chronic pain, or premenstrual syndrome. The invention, however, encompasses the use of the extracts in humans before, during and after treatment with prescription or conventional therapies.

In accordance with the present invention, the Rutaceae plant can be used alone or in combination with other known therapeutic agents or techniques to reduce anxiety, pain, chronic pain, depression, or premenstrual syndrome. Such agents may include vitamins and minerals, such as magnesium, calcium, anxiolytic agents, antidepressant agents, or analgesics.

Since novel formulations of Rutaceae plant are disclosed herein, the invention also encompasses methods of using the novel formulations for the treatment of anxiety, pain, chronic pain, depression, and premenstrual syndrome in a mammal, wherein the mammal is preferably a human or for the treatment of separation anxiety in domestic animals.

### 5.1 Method for Obtaining Rutaceae Plant Extracts

The plants belonging to the “Rutaceae family” used in the present invention are plants belonging to Phellodendron, Citrus, Evodia and Dictamnus. Phellodendron plants include, but are not limited to, Phellodendron amurense Ruprecht, Phellodendron amurense sachalinense, Phellodendron lavallei and Phellodendron chinense. Citrus plants include, but are not limited to, Citrus limon, Citrus sinensis, Citrus paradishi, Citrus aurantium, Citrus ichangensis, Citrus reticulata, Citrus x meyeri, Citrofortunella microcarpa, Fortunella japonica, Fortunella margarita, and Poncirus trifoliata. Evodia plants include, but are not limited to, Evodia rutaecarpa, Euodia fraxinifolia, Euodia lepta, and Euodia officinalis. Plants belonging to Dictamnus include, but are not limited to, Dictamnus albus.

The most preferable plants are Phellodendron amurense and its variants P. amurense Rupr. var. japonicum (Maxim.) ohwi, P. amurense Rupr. var. sachalinense Fr. Schm., P. ammurense Rupr. var. lavallei (Dode) Sprague, etc., P. chinenese Schneid and its variants, P. chinense Schneid. forma glabrinsculum (Schneid.) Hsiao, P. chinense Schneid. var. omeiense Huang, P. chinense Schneid. var. yunnanense Huang, P. chinenese Schneid. var. falcatum Huang, etc., P. wilsonii Hayata et Kanehira, Evodia rutaecarpa Hook. fil. et Thomson, Dictamnus albus L. subsp. dasycarpus KITAGAWA. In one embodiment, the plants exclude Murraya Koenigii.

The Rutaceae plant includes the plant parts as defined above, optionally the plant parts may be cut into small pieces or ground into a powder. Preferably, the plant part includes an extract of the Rutaceae plant. During a typical extraction process, the Rutaceae plant body, preferably cut into small pieces or ground into a powder, is placed in a Soxhlet extractor and extracted with any suitable solvent. Typical solvents include, but are not limited to, water, lower alcohols, or mixtures thereof. Preferably, the solvents used in the extraction include water, ethanol, and mixtures thereof. The solvent is maintained at reflux and the Rutaceae plant body is extracted for about 8 hours to about 48 hours. Preferably, the Rutaceae plant is extracted for about 12 hours to about 40 hours, and more preferably for about 18 hours to about 30 hours.

Subsequently, the solvent is separated and reduced in volume. Optionally, the solvent may be extracted with a second solvent. Thereafter, the extraction solvents are collected and reduced in volume either under low pressure or by evaporation to form a residue. Optionally, the residue is diluted and purified by gravity chromatography using at least one suitable solvent easily determined by a skilled artisan with little or no experimentation as the mobile phase. Optionally, the ratio of solvents within the solvent mixture may be gradually changed.

An alternative extraction process comprises adding a suitable solvent to the Rutaceae plant body, either grounded into a powder or cut into pieces. The solvents include, but are not limited to water, a lower alcohol, and mixtures thereof. Preferably, the solvents are water, ethanol, or mixtures thereof. The mixture of Rutaceae plant and solvent is allowed to sit overnight, preferably for about 6 hours to about 40 hours, preferably for about 8 hours to about 18 hours. Subsequently, the mixture is filtered, separating the solids from the filtrate. The solids are mixed with more solvent and allowed to sit overnight, preferably for about 6 hours to about 40 hours, preferably from about 12 hours to 32 hours, and more preferably from about 8 hours to about 18 hours. The mixture is separated a second time by filtration and the filtrates from both extractions are combined, and concentrated under reduced pressure to obtain a residue. The residue is vacuum dried for about 1 to about 10 hours, preferably for about 1 to about 2 hours at room temperature.

Yet another alternative extraction process comprises combining a suitable solvent to the Rutaceae plant body, either grounded into a powder or cut into pieces, in a ratio of about 4:1 to about 7:1 by volume to form a mixture. The mixture is heated to a temperature of about 1° F. below the boiling point of the solvent and stirred for about an hour. Preferably, if water is used as a solvent, the temperature is about 212° F. The mixture is filtered and the filtrate is washed with fresh solvent in a volume ratio of about 1:1. Subsequently the filtrate is concentrated under reduced volume and dried in a vacuum oven. In this method, suitable solvents include ethanol, methanol, chlornated solvents, propanol, 2-propanol, water, denatured industrial grade alcohol such as SDA-35, and mixtures thereof. Preferably, suitable solvents include water, ethanol, SDA-35, and mixtures thereof.

### 5.2 Compositions Comprising Rutaceae Plant Extracts and Modes of Administration

The invention comprises non-sedating compositions of Rutaceae plant or plant extracts with physiologically suitable carriers including, but not limited to, pharmaceutical carriers for the treatment of anxiety, pain, chronic pain, depression, and premenstrual syndrome. In a preferred embodiment, the compositions of the present invention are substantially free of lipophilic extracts of the Rutaceae plant, in particular obacunone and limonin. More preferably, the compositions include extracts obtained from the extraction of Rutaceae plant parts with an aqueous organic solution mixture, in particular a water and ethanol solution mixture, comprising small molecule compounds. In a more preferred embodiment, the compositions of the invention include water or water-organic alcohol soluble compounds including, but not limited to, berberine, phellodendrine, magnoflorine, and mixtures thereof. The magnitude of the therapeutic dose of an active ingredient in the acute or chronic management of a disorder or condition will vary with the severity of the disorder or condition to be treated and the route of administration. The dose, and perhaps the dose frequency, will also vary according to age, body weight, response, and the past medical history of the consumer or patient. Suitable dosing regimens can be readily selected by those skilled in the art with due consideration of such factors.

In one embodiment of the present invention, Rutaceae plant comprises about 2% to about 100% by weight of the composition. In a preferred embodiment, Rutaceae plant comprises about 5% to about 95% by weight of the composition. In a more preferred embodiment, Rutaceae plant comprises about 10% to about 90% by weight of the composition.

In another embodiment of the present invention using Rutaceae plant extracts, Rutaceae plant extracts comprise about 25% to about 100% by weight of the composition. In a preferred embodiment, Rutaceae plant extracts comprise about 35% to about 90% by weight of the composition. In a more preferred embodiment, Rutaceae plant extracts comprise about 40% to about 80% by weight of the composition.

In an embodiment, the composition of the invention comprises Rutaceae plant or plant extract present in an amount of about 35% to about 90% by weight and at least one anxiolytic agent. The anxiolytic agent includes, but is not limited to, barbituates, serotonin reuptake inhibitors (SSRIs), benzodiazepines, monoamine oxidase inhibitors, among others. Specifically, the anxiolytic agent may be selected from the group consisting of non-sedating agents such as Kava Kava and St. John's Wart, hydroxyzine hydrochloride, buspirone, mephobarbital, meprobamate, paroxetine, perphenazine, amitriptyline, fluvoxamine, sertraline, fluoxetine, alprazolam, chlordiazepoxide, clonazepam, clorazepate, diazepam, lorazepam, oxazepam, phenelzine, tranylcypromine, prazepam, halazepam, azapirones, gepirone, ipsopirone, tiaspirone, clozapine, fluperlapine, olanzapine, zotapine, seroquel, benzepine, preclamol pramipexole, emonapride, eticlopride, raclopride, remoxipride, hydroxyaminotetralins, hexahydrobenzophenanthridines, other dietary aids that have efficacy against anxiety, and mixtures thereof. Alternatively, the compositions of the invention can be administered sequentially or simultaneously in combination with at least one anxiolytic agent.

In an embodiment, the composition of the invention comprises Rutaceae plant or plant extract present in an amount of about 35% to about 90% by weight and at least one analgesic agent. The analgesic agent includes, but is not limited to, non-sedating agents, other dietary aids that have efficacy against pain and chronic pain, nonsteroidal anti-inflammatory drugs (NSAIDs), among others. Specifically, the analgesic agent is selected from the group consisting of aspirin, ibuprofen, ketoprofen, naproxen, acetaminophen, choline magnesium trisalicylate, diclofenac, diflunisal, fenoprofen, flurbiprofen, indomethacin, meclofenamate, nabumetone, oxaprozin, phenylbutazone, piroxicam, salsalate, sulindac, tolmetin, sodium salicylate, salicylsalicylic acid, sulfasalazine, olsalazine, etodolac, keterolac, mefenamic acid, meclofenamic acid, oxicams, piroxicam, tenoxicam, pyrazolidinediones, phenylbutazone, oxyphenthatrazone, and mixtures thereof. Alternatively, the compositions of the invention can be administered sequentially or simultaneously in combination with at least one analgesic agent.

In another embodiment, the composition of the invention comprises Rutaceae plant or plant extracts in an amount of about 35% to about 90% by weight and at least one anti-depressant agent. In this embodiment, the Rutaceae plant or plant extract and the anti-depressant agent act synergistically to enhance the anxiolytic and anti-depressant properties of the composition. The anti-depressant agent may be a pharmaceutical that has efficacy against depression, including, but not limited to, those selected from the group consisting of tricyclic antidepressants, monoanine oxidase inhibitors, and SSRIs, among others. Specifically, the anti-depressant agent may be selected from the group consisting of mirtazapine, nefazodone, bupropion, amitriptyline, amoxapine, clomipramine, desipramine, doxepin, imipramine, maprotiline, nortriptyline, protriptyline, trazodone, trimipramine, venlafaxine, paroxetine, perphenazine, amitriptyline, fluvoxamine, sertraline, fluoxetine, isocarboxazid, pargyline, phenelzine, tranylcypromine, dibenzazepines, selegiline, and mixtures thereof. Alternatively, the compositions of the invention can be administered sequentially or simultaneously in combination with at least one anti-depressant agent.

In an embodiment, the composition of the invention comprises Rutaceae plant or plant extract present in an amount of about 35% to about 90% by weight and at least one agent effective against premenstrual syndrome. The agent includes, but is not limited to, non-sedating agents, such as oral contraceptives which comprise estrogen and progestin; a mild diuretic, such as spironolactone; calcium, magnesium, vitamin B supplements, especially B₆ (pyroidoxine), and dietary aids that have efficacy against premenstrual syndrome. Alternatively, the compositions of the invention can be administered sequentially or simultaneously in combination with at least one agent against premenstrual syndrome.

Rutaceae plant or extracts thereof can be formulated using standard formulation techniques into gel caps, teas, tablets, etc. See, e.g. Remington's Pharmaceutical Sciences, 18th ed., Mack Publishing, Easton, Pa. (1990). Rutaceae plant extracts of the invention may be formulated into a dietary supplement or a pharmaceutical preparation for the administration to mammals for the treatment of anxiety, pain, chronic pain, depression, or premenstrual syndrome. In a preferred embodiment, the mammal is human.

Compositions comprising Rutaceae plant extracts of the invention formulated in a compatible pharmaceutical carrier may be prepared, packaged, and labeled for treatment, prevention, or management of anxiety, pain, chronic pain, depression, premenstrual syndrome, or symptoms thereof.

If the composition is water-soluble, then it may be formulated in an appropriate buffer, for example, phosphate buffered saline or other physiologically compatible solutions. Alternatively, if the resulting composition has poor solubility in aqueous solvents, then it may be formulated with a non-ionic surfactant such as Tween or olyethylene glycol. Thus, the compositions and their physiologically acceptable solvates may be formulated for administration by inhalation or insufflation (either through the mouth or the nose), oral, buccal, parenteral, or rectal administration.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art. In a preferred embodiment, the pharmaceutical composition may take the form of a capsule or powder to be dissolved in a liquid for oral consumption. Preparations for oral administration may be suitably formulated to give controlled release of the active compound.

For buccal administration, the compositions may take the form of tablets or lozenges formulated in conventional manner.

The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may comprise formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., comprising conventional suppository bases such as cocoa butter or other glycerides.

In addition to the formulations described previously, the compounds may also be a formulated as a sustained and/or timed release formulation. The compositions must be maintained above some minimum therapeutic dose to be effective. Such sustained and/or timed release formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs. Common timed and/or controlled release delivery systems include, but are not be restricted to, starches, osmotic pumps, or gelatin micro capsules.

The compositions may, if desired, be presented in a pack or dispenser device which may comprise one or more unit dosage forms comprising the active ingredient. The pack may for example comprise metal or plastic foil, such as a blister pack. The pack or dispenser device may be accompanied by instructions for administration.

### 5.3 Dietary Supplements Comprising Rutaceae Plant Extract

The compositions of the invention may include food compositions, over the counter, and dietary supplements. The Rutaceae plant or plant extract may be added to various foods so as to be consumed simultaneously. Preferably, the Rutaceae plant extract is substantially free of obacunone or limonin. As a food additive, the Rutaceae plant or plant extracts of the invention may be used in the same manner as conventional food additives, and thus, only needs to be mixed with other components to enhance the taste. Taste enhancement includes, but is not limited to, imparting to food a refreshingness, vitality, cleanness, fineness, or bracingness to the inherent taste of the food.

It will be recognized that dietary supplements may not use the same formulation ingredients or have the same sterile and other FDA requirements as pharmaceutical compositions. The dietary supplements may be in liquid form, for example, solutions, syrups or suspensions, or may be in the form of a product for reconstitution with water or any other suitable liquid before use. Such liquid preparations may be prepared by conventional means such as a tea, health beverage, dietary shake, liquid concentrate, or liquid soluble tablet, capsule, pill, or powder such that the beverage may be prepared by dissolving the liquid soluble tablet, capsule, pill, or powder within a liquid and consuming the resulting beverage. Alternatively, the dietary supplements may take the form of tablets or capsules prepared by conventional means and optionally including other dietary supplements including vitamins, minerals, other herbal supplements, binding agents, fillers, lubricants, disintegrants, or wetting agents, as those discussed above. The tablets may be coated by methods well-known in the art. In a preferred embodiment, the dietary supplement may take the form of a capsule or powder to be dissolved in a liquid for oral consumption.

The amount of Rutaceae plant or plant extract in a beverage or incorporated into a food product will depend on the kind of beverage, food and the desired effect. In general, a single serving comprises an amount of about 0.1% to about 50%, preferably of about 0.5% to about 20% of the food composition. More preferably a food product comprises Rutaceae plant or plant extract in an amount of about 1% to about 10% by weight of the food composition.

Examples of food include, but are not limited to, confectionery such as sweets (candies, jellies, jams, etc.), gums, bean pastes, baked confectioneries or molded confectioneries (cookies, biscuits, etc.), steamed confectioneries, cacao or cacao products (chocolates and cocoa), frozen confectioneries (ice cream, ices, etc.), beverages (fruit juice, soft drinks, carbonated beverages), health drinks, health bars, and tea (green tea, black tea, etc.).

### 5.4 Veterinarian Applications

The compositions of the invention include veterinarian compositions directed to the treatment, prevention, or management of diseases or disorders including separation anxiety in domestic animals. The Rutaceae plant or plant extract may be added to various pet foods or medications so as to be consumed simultaneously by a domestic animal. When combined with pet food, the Rutaceae plant or plant extracts of the invention may be used in the same manner as conventional food additives, and thus, only needs to be mixed with other components. When combined with pet medications, the Rutaceae plant or plant extract may be combined as described above for pharmaceutical compositions. Preferably, the Rutaceae plant extract is substantially free of obacunone or limonin.

In one embodiment of the present invention, the composition for treating separation anxiety in domestic animals comprises Rutaceae plant or plant extract in sufficient amount to prevent the onset of separation anxiety and separation anxiety related symptoms. If the domestic animal already suffers from separation anxiety, the composition should comprise sufficient amount of Rutaceae plant or Rutaceae plant extract to reduce separation anxiety. In general, a single serving comprises an amount of about 0.1% to about 50%, preferably of about 0.5% to about 20% by weight of the pet food composition. More preferably, a pet food product comprises Rutaceae plant or plant extract in an amount of about 1% to about 10% by weight of the pet food composition.

### 5.5 Dosage

The magnitude of a therapeutic dose of Rutaceae plant in the prevention, treatment, or management of acute or chronic anxiety, pain, chronic pain, depression, premenstrual syndrome, weight loss, cold, appetite loss, sleeplessness, or fatigue will vary with the severity of the condition of the consumer or patient to be treated and the route of administration. The dose, and dose frequency, will also vary according to the age, body weight, condition and response of the individual consumer or patient, and the particular Rutaceae plant combination used. All combinations described in the specification are encompassed as therapeutic, and it is understood that one of skill in the art would be able to determine a proper dosage of particular Rutaceae plant composition using the parameters provided in the invention.

In general, the total daily dose ranges of the Rutaceae plant for the conditions described herein are generally from about 7 mg/kg to about 140 mg/kg administered in divided doses administered parenterally, orally, or topically. A preferred total daily dose is from about 15 mg/kg to about 130 mg/kg of the Rutaceae plant composition. When a Rutaceae plant extract is used, the total daily dose ranges of the Rutaceae plant extract for the conditions described herein are generally from about 4 mg/kg to about 13 mg/kg administered in divided doses administered parenterally, orally, or topically. A preferred total daily dose is from about 5 mg/kg to about 11 mg/kg of the Rutaceae plant extracts.

For example, in one embodiment, the daily dose ranges of Rutaceae plant extracts compositions described herein are generally about 7.0 mg per body weight of Rutaceae plant extracts composition. Preferably the Rutaceae plant extracts formulation of the invention is given daily until the symptoms cease, followed by two to ten additional cycles, each lasting about 60 days in duration. When the dose is administered orally, a sustained release formulation is preferred so that a fairly constant level of Rutaceae plant extracts is provided over the course of treatment. As the Rutaceae plant extracts are not particularly toxic, the formulation may be administered for as long as necessary to achieve the desired therapeutic effect.

In the case where an intravenous injection or infusion composition is employed, a suitable dosage range for use is, e.g., from about 0.01 to about 150 mg per kg body weight of Rutaceae plant extracts daily.

Again, any suitable route of administration may be employed for providing the consumer or patient with an effective dosage of Rutaceae plant or plant extracts composition of this invention. Dosage forms include tablets, troches, cachet, dispersions, suspensions, solutions, capsules, gel caps, caplets, compressed tablets, sustained release devices, patches, and the like.

The dietary supplements and pharmaceutical compositions of the present invention comprise Rutaceae plant or plant extracts as the active ingredients, as well as pharmaceutically acceptable salts thereof, and may also comprise a pharmaceutically acceptable carrier, and optionally, other therapeutic ingredients.

The dietary supplements and pharmaceutical compositions include compositions suitable for oral and parenteral (including subcutaneous, intramuscular, intrathecal, intravenous, and other injectables) routes, although the most suitable route in any given case will depend on the nature and severity of the condition being treated.

In addition, the Rutaceae plant or plant extracts carrier could be delivered via charged and uncharged matrices used as drug delivery devices such as cellulose acetate membranes, also through targeted delivery systems such as liposomes attached to antibodies or specific antigens.

In practical use, Rutaceae plant or plant extracts can be combined as the active ingredient(s) in intimate admixture with a pharmaceutical carrier according to conventional pharmaceutical compounding techniques. The carrier may take a wide variety of forms depending on the form of preparation desired for administration, e.g., oral or parenteral (including tablets, capsules, powders, intravenous injections or infusions). In preparing the compositions for oral dosage form any of the usual pharmaceutical media may be employed, e.g., water, glycols, oils, alcohols, flavoring agents, preservatives, coloring agents, and the like; in the case of oral liquid preparations, e.g. suspensions, solutions, elixirs, liposomes and aerosols; starches, sugars, micro-crystalline cellulose, diluents, granulating agents, lubricants, binders, disintegrating agents, and the like in the case of oral solid preparations e.g., powders, capsules, and tablets. In preparing the compositions for parenteral dosage form, such as intravenous injection or infusion, similar pharmaceutical media may be employed, e.g., water, glycols, oils, buffers, sugar, preservatives, and the like know to those skilled in the art. Examples of such parenteral compositions include, but are not limited to Dextrose 5% (w/v), normal saline or other solutions. The total dose of the Rutaceae plant extracts may be administered in a vial of intravenous fluid, e.g. ranging from about 0.7 to about 14 mg per kg body weight of Rutaceae plant extracts. The volume of dilution fluid will vary according to the total dose administered and over the length of the period of time of administration.

### 6. EXAMPLES

Certain embodiments of the invention, as well as certain novel and unexpected advantages of the invention, are illustrated by the following non-limiting examples.

### 6.1 Anti-Anxiety Activity

### 6.1.1 Materials and Methods

Using accepted chick model experiments, the extracts of the invention were tested for their anti-anxiety properties as discussed below.

### 6.1.1.1 Subjects

Cockerels (Gallus gallus; W36 strain, Cal-Maine Foods Inc., Mendenhall, Miss.) were obtained 1-day post-hatch and group housed in stainless steel cages at 12 chicks per cage. Food (Purina Start and Grow, Ralston-Purina Company, Checkerboard Square, St. Louis, Mo. 63164) and water were available ad libitum. Room temperature was maintained at 29+1° C., and overhead illumination was maintained on a 12:12 h light dark cycle. Chicks were handled briefly each day during daily maintenance prior to testing in order to reduce experimenter-related stress (Sufka, K. J., Hughes, R. A., “Differential effects of handling on isolation-induced vocalizations, hypoalgesia, and hyperthermia in domestic fowl,” Physiology and Behavior, 1991, 50, pp. 129-133). The Institutional Animal Care and Use Committee approved all research protocols, and studies were conducted under the ethical guidelines specified by the Animal Welfare Act and National Institute of Health (NIH).

### 6.1.1.2 Apparatus

Three Plexiglas viewing chambers (25×25×22 cm) situated in separate sound attenuating boxes were used for data collection (Sufka, K. J., Weed, N. C., “Construct validation of behavioral indices of isolation stress and inflammatory nociception in young domestic fowl,” Physiology and Behavior, 1994, 55, pp. 741-746). Distress vocalizations (DVoc) were monitored via microphones located above the observation chambers, which were connected to sound activated relays that triggered counters.

### 6.1.1.3 Test Materials

The following samples were developed and screened for anxiolytic activity:

Formulation A: a hydroalcoholic extract from the Rutaceae family (a composition of the invention);

Formulation B: an aqueous extract from the Rutaceae family (a composition of the invention);

Formulation C: a hydroalcoholic extract of Acori graminei;

Formulation D: an aqueous extract of Acori graminei;

Formulation E: an aqueous extract from the Magnoliaceae family;

Formulation F: a hydroalcoholic extract from the Magnoliaceae family;

Formulation G: a hydroalcoholic extract of Ladies Mantle; and

Formulation H: an alcoholic extract of Primula veris.

The vehicle for all preparations was: 40 ml propylene glycol, 10 ml ethanol, and 50 ml water. All samples were tested at a concentration of 25 mg/ml with the exception of samples Formulation B and Formulation G, which were tested at a concentration of 28 mg/ml.

### 6.1.1.4 Procedure

All tests were conducted at 8-days post-hatch. The first experiment was to determine whether various botanical extracts possessed anxiolytic properties. Single doses of these extracts were tested in groups of 4 per session with 2 screening sessions in this experiment. The design for each screening session formed a single factorial design (vehicle plus four compounds in isolated chicks) with a hanging control (vehicle treated in social testing condition). Sample sizes were n=18 per group. Drug injections (or vehicle) were administered (1.0 ml/kg) IP 30 min before testing. In a second experiment, two dose response sessions were conducted in which one test article was screened per session. Samples or vehicle were administered in a volume of 2.0 ml/kg IP 30 min before tests. Drugs doses were 12.5, 25, and 50 mg/2 ml/kg for Formulation F and 14, 28, and 56 mg/2 ml/kg for Formulation B. The experimental design for both dose response sessions formed a 2×4 factorial which combined two levels of stress (social vs. isolated) with 4 levels of dose (vehicle and 3 doses). Sample sizes were n=15 per group. The stress manipulation involved placing a chick into the observation chamber in isolation or in the presence of two conspecifics (social). To index stress-induced analgesia (SIA), chicks received 50 μl of 0.10% formalin into the plantar region of its foot immediately before placement in the chamber. Trained observers recorded footlift frequency and footlift duration in response to formalin and latency to adopt a sleep-like posture, i.e., ventral recumbent latency (VRL) to index sedation. The observation session was 180 s in duration. A composite pain score (CPS) was derived from the following formula: CPS 4(z-score footlift)+(z-score duration/total number of lifts) (Roach, J. T., Watson, G. S., Sufka, K. J., “Optimal scoring strategy of the domestic fowl chick formalin test,” Society for Neuroscience Abstract, 1998, 1, pp. 453). Data were screened for homogeneity of variance and analyzed using analysis of variance (ANOVA) and Tukey procedures (Kirk, R. E., “Experimental Design: Procedures for the behavioral sciences,” Brooks Cole Publishing Corporation, 1982, Monterey).

### 6.1.2 Results

### 6.1.2.1 Screening Session 1

This test session sought to determine whether Formulation H, Formulation E, Formulation C, and Formulation A possessed anxiolytic effects in the chick social separation-stress procedure. As is evident in Table 1, VRL measures were not affected by any of the four test articles; nearly all animals across groups remained active through the entire observation period. These data indicate that samples Formulation H, Formulation E, Formulation C, and Formulation A do not possess sedative properties at the doses tested.

The results from the vocalization measure are summarized in Table 1. Vehicle-isolated chicks exhibited significantly more DVoc than vehicle-treated chicks tested in the social condition, illustrating the social separation stress effect, t(33)=4.70, p<0.01. At the doses tested, Formulation H, Formulation E, and Formulation C elevated DVoc and Formulation A did not alter DVoc. These data indicate that all four test articles in this screening session do not possess anxiolytic effects on this measure.

TABLE 1
Effects of Botanical Extracts in the Chick Social-Separation
Stress Procedure
Dependent Measures

| | VRL | DVoc | CPS |
|---|---|---|---|
| Session 1 | | | |
| Social-Vehicle | 172.28 (3.62) | 10.78 (5.53) | 2.41 (1.28) |
| Isolated Vehicle | 179.47 (0.53) | 74.53 (12.69) | 0.66 (0.90) |
| Formulation H | 173.75 (4.07) | 104.06 (24.41) | −0.48 (0.76) |
| Formulation E | 180.00 (0.00) | 109.89 (17.33) | −1.14 (0.61) |
| Formulation C | 176.41 (3.59) | 113.59 (15.29) | −1.19 (0.74) |
| Formulation A | 179.94 (0.06) | 80.00 (24.01) | −0.82 (0.81) |
| Session 2 | | | |
| Social-Vehicle | 167.69 (6.10) | 6.88 (2.12) | 4.09 (1.43) |
| Isolated-Vehicle | 174.07 (5.26) | 125.60 (20.65) | −0.37 (0.84) |
| Formulation G | 174.56 (4.99) | 98.56 (17.98) | −0.77 (0.60) |
| Formulation F | 171.61 (4.93) | 62.00 (16.17) | −1.36 (0.70) |
| Formulation D | 170.38 (5.24) | 104.75 (17.92) | −0.78 (0.65) |
| Formulation B | 179.86 (0.14) | 60.79 (17.31) | −0.60 (0.68) |

Note:
Values represent group means (standard error in parenthesis).

The results from the nociceptive measures used to index stress-induced analgesia (SIA) are summarized in Table 1. Vehicle-isolated chicks had a lower CPS than chicks tested in the vehicle-social condition, a pattern consistent with SIA. However, large variability prevented detection of any statistically significant difference among groups. All four of the test articles lowered the CPS. These data indicate that Formulation H, Formulation E, Formulation C, and Formulation A do not possess anxiolytic effects at this dosage. Taken collectively, results from this screening session suggest Formulation H and Formulation A do not alter chick stress responses while Formulation E and Formulation C, if anything, enhanced chick stress responses in this paradigm.

### 6.1.2.2 Screening Session 2

This test session sought to determine whether Formulation G, Formulation F, Formulation D, and Formulation B possessed anxiolytic effects in the chick social separation-stress procedure. As is evident in Table 1, VRL measures were not affected by any of the four test articles; nearly all animals across groups remained active throughout the entire observation period. These data indicate that Formulation G, Formulation F, Formulation D, and Formulation B do not possess sedative properties at the doses tested.

The results from the vocalization measure are summarized in Table I. In the vehicle groups, isolated chicks vocalized significantly more than chicks tested in the social condition, illustrating the social separation stress effect, t(29)=5.91, p<0.01. At the doses tested, Formulation F and Formulation B reversed the social separation stress effect of DVoc to approximately 50% of control, t(31)=2.46, p<0.05 and t(27)=2.39, p<0.05, respectively. Formulation G and Formulation D did not affect DVoc rates. These data suggest that at the doses tested, Formulation F and Formulation B possessed anxiolytic effects on this measure.

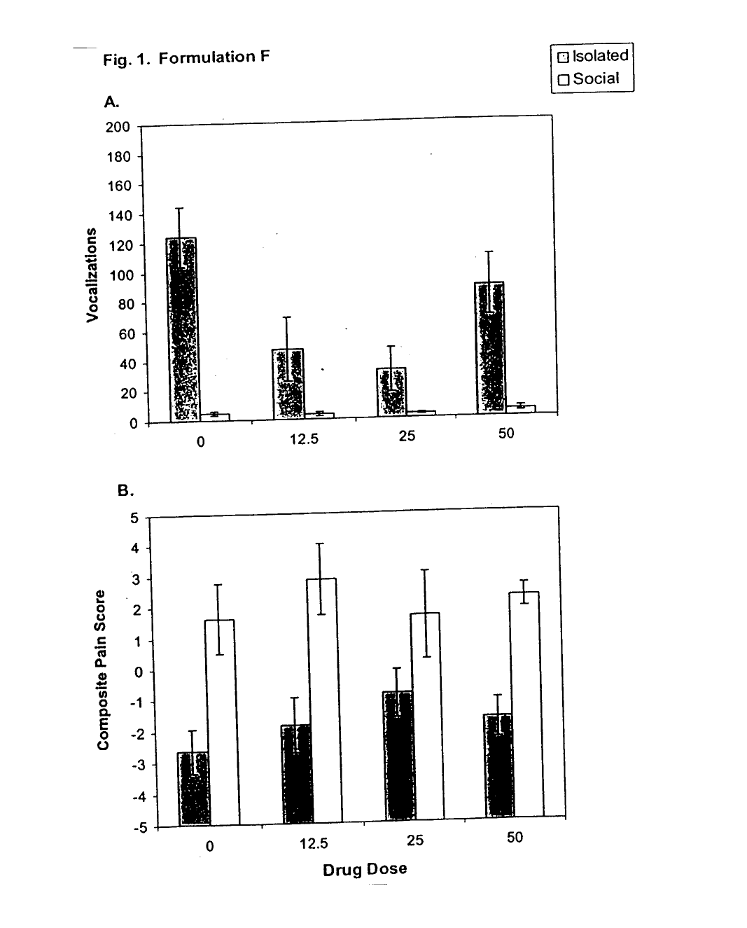

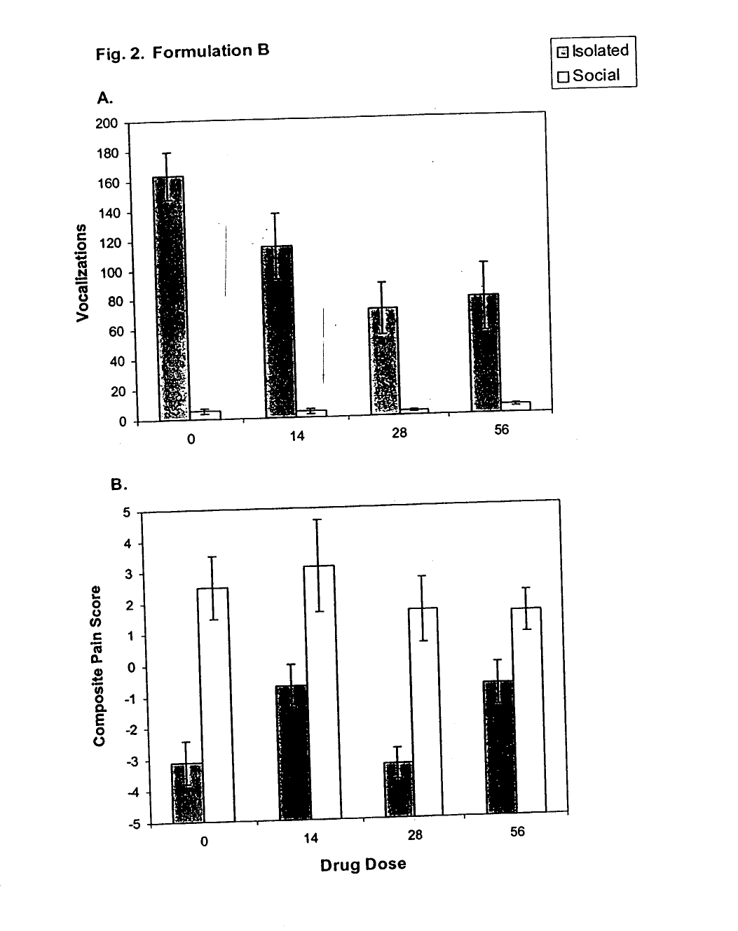

The results from the nociceptive measures used to index SIA are summarized in Table I. In vehicle treated groups, isolated chicks had a significantly lower CPS than chicks tested in the social condition, illustrating the social separation stress effect, 1(29)=-2.64, p<0.05. None of the four test articles significantly affected the CPS. These data indicate that, at the doses tested, Formulation G, Formulation F, Formulation D, and Formulation B did not possess anxiolytic effects at this dosage.

Whereas Formulation B and Formulation F attenuated distress vocalizations, only Formulation B attenuated stress-induced analgesia. See FIGS. 1B and 2B. This result is consistent with the effect of various benzodiazepine anxiolytics in the model study (Watson, G. S., Sufka, K. J., “Chlordiazepoxide reverses social-separation-induced distress vocalizations and analgesia in young domestic fowl,” Experimental and Clinical Psychopharmacology, 1996, 4, pp. 347-353; Watson, G. S., Roach, J. T., Sufka, K. J., “Benzodiazepine receptor function in the chick social-separation-stress procedure,” Experimental and Clinical Psychopharmacology, 1999, 7, pp. 83-89). Separation-induced vocalizations were more sensitive to anxiolytic manipulations than the nociceptive measures used to index stress-induced analgesia (Watson et al., 1999). Compounds that additionally possessed analgesic properties affected the nociceptive measure by masking a drug's anxiolytic effect. These factors may account for the absence of an effect by Formulation F and the variable effects by Formulation B in this paradigm.

Previous research with this chick model determined ED₅₀ values for the benzodiazepine agonists chlordiazepoxide, lorazepam, and alprazolam in attenuating separation-induced DVoc. The values were 3.75 mg/kg for chlorodiazepoxide (Watson et al., 1996), 0.34 mg/kg for lorazepam, and 0.19 mg/kg for alprazolam (Watson et al., 1999). The ED₅₀ values were well within their respective daily recommended dose ranges for humans (Baldessarini, R. J., “Drugs and the treatment of psychiatric disorders: Psychosis and anxiety,” The pharmacological basis of therapeutics, 1996, Hardman, J. G., Gilman, A. G., Limbird, L. E., eds., 9^{th} ed., McGraw-Hill, New York, pp. 399-430). Calculation of accurate ED₅₀ values in the present study was difficult to determine because of the limited number of screened doses of Formulation B and Formulation F. The vocalization measure response pattern, however, suggested approximate ED₅₀ of 1 mg/kg and 12 mg/kg for Formulation F and Formulation B, respectively. These findings prompted a second experiment in which dose-response characteristics of Formulation F and Formulation B were evaluated in the chick social-separation-stress procedure.

### 6.1.2.3 Formulation F Dose-Response

Formulation F did not affect VRL at any of the three doses tested in either the isolated or social tested chicks (see Table 2). These observations are consistent with findings from Experiment 6.1.2.2 and indicate that Formulation F does not possess sedative properties within this dose range.

The results of Formulation F on DVoc are summarized in FIG. 1A. In vehicle control groups, isolated chicks (closed bar) vocalized significantly more than chicks tested under the social condition, illustrating the social separation stress effect, t(26)=5.56, p<0.05. In the isolated groups, Formulation F produced a dose-dependent reduction in vocalizations, F(3.49)=4.65, p<0.01, with the intermediate dose producing the largest effect. Formulation F did not alter DVoc in the social control groups; this is not unexpected due to the floor effect of this measure in social-tested chicks. These data are consistent with the notion that Formulation F possesses anxiolytic effects on this stress measure. It is difficult to derive an accurate ED₅₀ on this measure because of the limited number of doses tested in this trial. However, the pattern of response suggests an ED₅₀ of approximately 10 mg/ml delivered 2 ml/kg.

The results of Formulation F on CPS, an index of SIA, are summarized in FIG. 1B. In vehicle control groups, isolated chicks (closed bar) had a significantly lower CPS than chicks tested under the social condition, illustrating the social separation stress effect, t(24)=−3.16, p<0.01. This pattern is indicative of SIA produced by social separation. In the isolated groups, Formulation F produced a modest, although not statistically significant, attenuation of SIA (i.e., an increase in CPS), particularly at the intermediate dose. A similar effect was detected at the lowest dose of Formulation F in socially-tested chicks. The observation of anxiolytic effects under the social condition is possible when one considers that even these animals are likely subject to some stress during test procedures. These data suggest that Formulation F possesses only modest anxiolytic effects on this stress measure. Taken collectively, these results demonstrate that Formulation F tested positive in the chick social-separation stress paradigm for anxiolytic effects.

### 6.1.2.4 Formulation B Dose-Response

Formulation B did not affect the ventral recumbent measure at any of the three doses tested in either the isolated or social tested chicks (see Table 2). These observations are consistent with findings from Experiment 6.1.2.2 and indicate that Formulation B does not possess sedative properties within this dose range.

TABLE 2
VRL Measures for Formulation F and Formulation B
Dose

| | Vehicle | | Low | | Medium | | High | |
|---|---|---|---|---|---|---|---|---|
| Sample | Social | Isolated | Social | Isolated | Social | Isolated | Social | Isolated |
| Formulation F | 167.07 | 174.80 | 151.21 | 166.62 | 177.36 | 171.53 | 168.71 | 180.00 |
| | (6.93) | (5.32) | (10.00) | (5.23) | (2.00) | (4.87) | (5.50) | (0.00) |
| Formulation B | 172.67 | 174.67 | 170.25 | 176.53 | 166.20 | 169.73 | 169.87 | 173.31 |
| | (5.11) | (5.33) | (5.28) | (3.47) | (6.65) | (7.07) | (4.84) | (5.13) |

Note:
Values represent group means (standard errors in parentheses).

The results of Formulation B on DVoc are summarized in FIG. 2A. In vehicle control groups, isolated chicks (closed bar) vocalized significantly more than chicks tested under the social condition, illustrating the social separation stress effect, 1(28)=9.86, p<0.01. In the isolated groups, Formulation B produced a significant dose-dependent reduction in DVoc, F(3.54)=5.45, p<0.01, with the two highest doses producing the largest effect. Formulation B did not alter DVoc in the social control groups; as discussed above, this is not unexpected due to the floor effect of this measure in social-tested chicks. These data are consistent with the notion that Formulation B possesses anxiolytic effects on this stress measure. Again, it is difficult to derive an accurate ED₅₀ on this measure because of the limited number of doses tested in this trial. However, the pattern of response suggests an ED₅₀ of approximately 12 mg/ml delivered 2 ml/kg.

The results of Formulation B on CPS are summarized in FIG. 2B. In vehicle control groups, isolated chicks (closed bar) had significantly lower composite pain scores (CPS) than chicks tested under the social condition, illustrating the social separation stress effect, 1(26)=−4.58, p<0.01. In the isolated groups, Formulation B produced a significant attenuation of SIA (i.e., an increase in CPS), F(3.54)=4.35, p<0.01, particularly at the lowest and highest doses tested. Formulation B did not affect the CPS in social tested chicks. These data demonstrate that Formulation B possesses anxiolytic effects on this stress measure. Taken collectively, these results demonstrate that Formulation B proved to test positive in the chick social-separation stress paradigm for anxiolytic effects.

### 6.2 Analgesic Properties

### 6.2.1 Materials and Methods

The analgesic effects of the extracts of this invention were studied using the formalin test (Dubuisson, D., Dennis, S. G., “The formalin test: a quantitative study of the analgesic effects of morphine, meperidine, and brainstem stimulation in rats and cats,” Pain, 1977, 4, pp. 161-164), a commonly used animal model to study nociceptive processes and analgesic drug effects.

### 6.2.1.1 Subjects

Male Holtzman rats weighing 150-175 g were obtained from Harlan Spraque Dawley (Indianapolis, Ind.) and housed under a 12:12 h light/dark cycle in individual stainless steel cages in a temperature and humidity controlled vivarium. Food and water were available ad libitum. In session one, the animals were allowed to acclimate to the colony room for two days and handled for an additional two days prior to conducting the experiment in order to reduce experimenter-related stress. In session two, animals were allowed to acclimate to the colony room for four days, handled for three days, and acclimated to the apparatus for two days prior to conducting the experiment in order to reduce experimenter-related stress. In both sessions, the animals were food-deprived for 12 h before administering the test articles.

### 6.2.1.2 Apparatus

A Plexiglas viewing chamber (30×30×30 cm) was used for data collection.

### 6.2.1.3 Test Materials

The following samples were developed and screened for analgesic activity:

Formulation K: berberine HCl

Formulation J: an aqueous extract from the Rutaceae plant family and berberine HCl.

Naproxen was used as a reference. Samples were tested at a concentration of 100 mg/kg except Formulation J which was tested at 200 mg/kg.

In session one, 10 minutes before the formalin observation period, the formulations were delivered via gavage in a volume of 0.5 ml to obtain a minimum response interval of 30 min between the injection time and the second phase of the formalin response. In session two, formulations were delivered via gavage of 1 ml. Each animal received two formulation dosings before the tests: 70 and 10 minutes before the beginning of the formalin observation period in order to obtain a 90 and 30 min injection intervals before the second phase of the formalin response.

### 6.2.1.4 Procedure

Immediately after administration of 0.05 ml of 2.5% formalin into the hindpaw, rats were placed into 30×30×30 cm Plexiglas observation chamber. The observation session lasted 50 min. A dependent measure was the amount of time in which the injected paw was 1) in a lift position and 2) licked, bitten or shaken across ten 5-min blocks. A composite pain score as a function of ten 5-min blocks was calculated according to the procedures of Watson et al. (Watson, G. S., Sufka, K. J., Coderre, T. J., “Optimal scoring strategies and weights for the formalin test in rats,” Pain, 1997, 70, pp. 53-58). Sample sizes were n=5-6 rats per group. Each animal was tested twice; the second formalin test (in the contralateral paw) was conducted two days after the first.

Data were screened for homogeneity of variance and analyzed using repeated measures analysis of variance (ANOVA) and Fisher's LSD procedures.

### 6.2.2 Results

### 6.2.2.1 Session 1

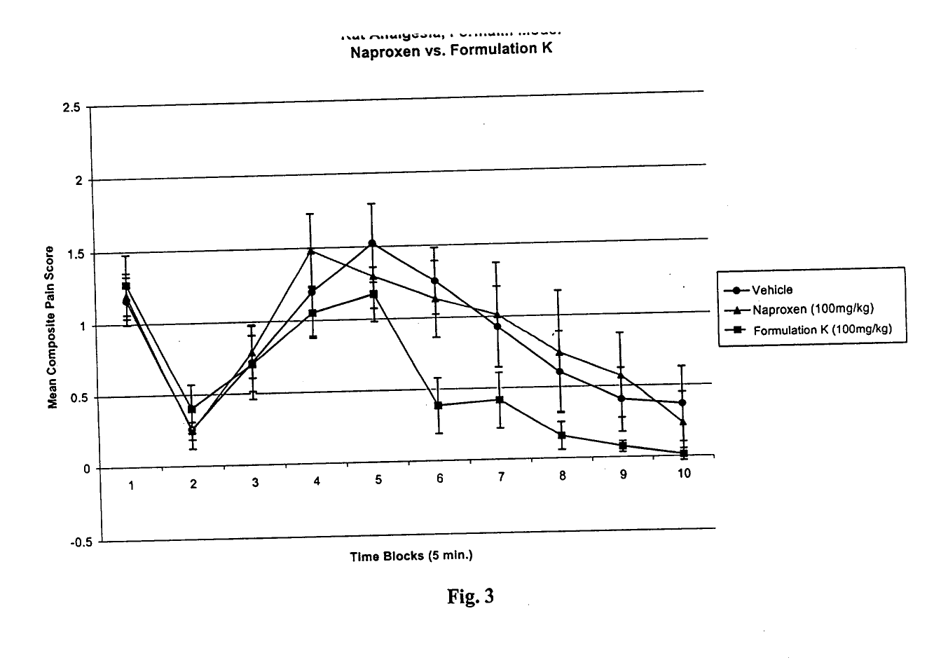

The results from the screening tests on Naproxen and Formulation K are summarized in FIG. 3. In vehicle treated rats, formalin induced a biphasic pain response. This response was characterized by an increase in pain-related behaviors within the 1st time block (Phase 1), a decrease in pain-related behaviors during the 2nd time block, and an increase in pain-behaviors during time blocks 4 through 8 (Phase 2). This pattern of pain response was consistent with earlier reports of formalin-induced nociception in rats. Naproxen did not affect pain behaviors in either the first or second phases of the formalin response. These data suggest Naproxen, at the dose tested, was ineffective in modulating inflammatory nociception in this model.

Formulation K attenuated pain-related behaviors during the second phase of the formalin response. These data suggest that Formulation K possessed analgesic properties in this model at the 100 mg/kg dose and the injection-to-test interval used.

These screening trials demonstrate that only Formulation K (100 mg/kg) possessed analgesic effects in the formalin test in rats. The data further demonstrated that at equal doses naproxen had no effect on pain whereas Formulation K reduced pain during the fourth to the tenth time block.

6.2.2.2 Session 2

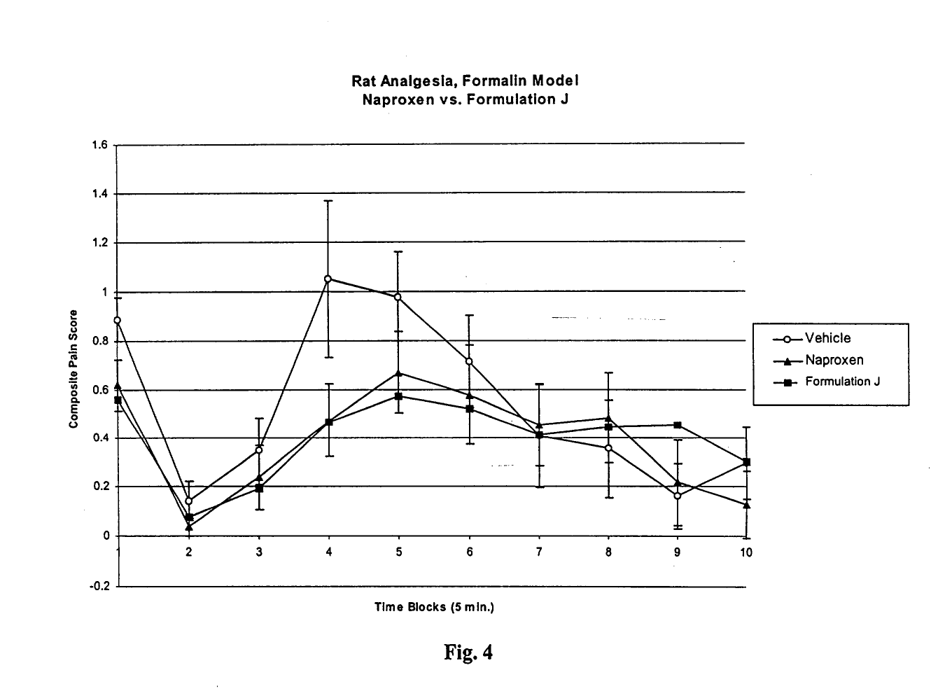

The results from the screening tests on Naproxen and Formulation J are summarized in FIG. 4. In vehicle treated rats (n=5), formalin induced a biphasic pain response as described in 6.2.2.1. Naproxen attenuated the second phase of the formalin response, as determined by the composite pain scores (CPS). These data indicate that the 100 mg dose of Naproxen is effective in modulating inflammatory nociception in this model.

Formulation J attenuated pain-related behaviors during the second phase of the formalin response. These data indicated that for this model that Formulation J possessed analgesic properties at the 100 mg/kg dose and the injection-to-test interval. Comparing the CPS measures from this study and the Naproxen data set indicated that Formulation J possessed equivalent analgesic properties to that of Naproxen.

### 6.3 Bioassay Results

### 6.3.1 Materials and Methods

Using standard methodologies, commercially available binding assays were used to determine binding affinity for a particular receptor. The sample concentrations were typically about 100 μg/ml. The results of the assays are summarized in Table 3. The results are reported as a percentage inhibition. Typically, the baseline runs from about −20% to about +20% inhibition of binding enzyme activity, within this range the compounds are considered to be inactive. A range from about 20% to about 49% shows marginal activity at the receptor site and generally does not warrant further examination. A range of about 50% and greater indicates the compound is active.

### 6.3.1.2 Pharmacological Screenings

Formulation A and Formulation B demonstrated significant binding to GABA_{A} and serotonin transporter in central nervous system binding assays. GABA_{A} is believed to be involved in anxiety. (Huong, N.; Matsumoto, K.; Yamasaki, K.; and Watanabe, H., “Majonoside-R2 reverses social isolation stress-induced decrease in pentobarbital sleep in mice; Possible involvement of neuroactive steroids,” Life Sciences, 1997, 61(4), pp. 395-402). Formulation B and Formulation F also had significant binding to glutamate NMDA and had little binding to the benzodiazepine receptors that might cause sedation. Consequently, Formulation B and Formulation F were not expected to cause sedation.

### 6.3.2 Results by Assay

Table 3 illustrates the binding affinity results obtained with the formulations previously discussed.

Formulation I: an alcoholic extract of Phellodendron.

TABLE 3

| Assay Name | Formulation | Result |
|---|---|---|
| Adenosine Al | Formulation A | 60.34 |
| Adenosine Al | Formulation A | 48.85 |
| Adenosine Al | Formulation B | 67.82 |
| Adenosine Al | Formulation B | 31.65 |
| Adenosine Al | Formulation C | 38.01 |
| Adenosine Al | Formulation E | 25.57 |
| Adenosine Al | Formulation F | 75.94 |
| Adenosine Al | Formulation G | 19.19 |
| Adenosine Al | Formulation H | 23.42 |
| Adenosine Al | Formulation I | 94.85 |
| Adrenergic Alpha1 Non-Selective | Formulation A | 76.27 |
| Adrenergic Alpha1 Non-Selective | Formulation B | 81.25 |
| Adrenergic Alpha1 Non-Selective | Formulation C | −21.61 |
| Adrenergic Alpha1 Non-Selective | Formulation E | −55.99 |
| Adrenergic Alpha1 Non-Selective | Formulation F | −116.7 |
| Adrenergic Alpha1 Non-Selective | Formulation G | −123 |
| Adrenergic Aipha1 Non-Selective | Formulation H | −124.5 |
| Adrenergic Alpha2 Non-Selective | Formulation A | −7.65 |
| Adrenergic Alpha2 Non-Selective | Formulation A | 89.77 |
| Adrenergic Alpha2 Non-Selective | Formulation B | 90.34 |
| Adrenergic Alpha2 Non-Selective | Formulation B | 76.47 |
| Adrenergic Alpha2 Non-Selective | Formulation C | 47.76 |
| Adrenergic Alpha2 Non-Selective | Formulation E | 2.97 |
| Adrenergic Alpha2 Non-Selective | Formulation F | 49.69 |
| Adrenergic Alpha2 Non-Selective | Formulation G | 14.73 |
| Adrenergic Alpha2 Non-Selective | Formulation H | 29.17 |
| Adrenergic Alpha2 Non-Selective | Formulation I | 78.81 |
| Bradykinin2 | Formulation A | 23.59 |
| Bradykinin2 | Formulation F | 12.09 |
| CCKB Cholecystokinin | Formulation A | −0.67 |
| CCKB Cholecystokinin | Formulation B | 2.08 |
| CCKB Cholecystokinin | Formulation I | 15.76 |
| Corticotropin Releasing Factor | Formulation A | −21.14 |
| Corticotropin Releasing Factor | Formulation B | −29.93 |
| Corticotropin Releasing Factor | Formulation C | −11.79 |
| Corticotropin Releasing Factor | Formulation E | −1.5 |
| Corticotropin Releasing Factor | Formulation F | −52.03 |
| Corticotropin Releasing Factor | Formulation G | 20.25 |
| Corticotropin Releasing Factor | Formulation H | −49.4 |
| GABA_{A} Agonist Site | Formulation A | 97.07 |
| GABA_{A} Agonist Site | Formulation A | 104.85 |
| GABA_{A} Agonist Site | Formulation B | 93.43 |
| GABA_{A} Agonist Site | Formulation B | 95.91 |
| GABA_{A} Agonist Site | Formulation C | 107.9 |
| GABA_{A} Agonist Site | Formulation E | 77.6 |
| GABA_{A} Agonist Site | Formulation F | 77.73 |
| GABA_{A} Agonist Site | Formulation G | 108.71 |
| GABA_{A} Agonist Site | Formulation H | 109.94 |
| GABA_{A} Agonist Site | Formulation I | 20.3 |
| GABA_{A} Benzodiazepine Central | Formulation A | 29.48 |
| GABA_{A} Benzodiazepine Central | Formulation A | −49.89 |
| GABA_{A} Benzodiazepine Central | Formulation B | −25.77 |
| GABA_{A} Benzodiazepine Central | Formulation B | 19.75 |
| GABA_{A} Benzodiazepine Central | Formulation C | 12.28 |
| GABA_{A} Benzodiazepine Central | Formulation E | −11.35 |
| GABA_{A} Benzodiazepine Central | Formulation F | 8.9 |
| GABA_{A} Benzodiazepine Central | Formulation G | 8.34 |
| GABA_{A} Benzodiazepine Central | Formulation H | 27 |
| GABA_{A} Benzodiazepine Central | Formulation I | −39.39 |
| GABA Chloride, TBOB Site | Formulation A | 52.89 |
| GABA Chloride, TBOB Site | Formulation B | −6.24 |
| GABA Chloride, TBOB Site | Formulation I | 74.9 |
| Glucocortoid | Formulation A | 21.83 |
| Glucocortoid | Formulation F | 18.9 |
| Glutamate AMPA Site | Formulation A | 27.72 |
| Glutamate AMPA Site | Formulation B | 41.88 |
| Glutamate AMPA Site | Formulation C | 52.96 |
| Glutamate AMPA Site | Formulation E | 45.89 |
| Glutamate AMPA Site | Formulation F | 16.39 |
| Glutamate AMPA Site | Formulation G | 21.19 |
| Glutamate AMPA Site | Formulation H | 69.32 |
| Glutamate NMDA Agonist Site | Formulation A | 73.79 |
| Glutamate NMDA Agonist Site | Formulation A | 26.51 |
| Glutamate NMDA Agonist Site | Formulation B | 18.97 |
| Glutamate NMDA Agonist Site | Formulation B | 59.15 |
| Glutamate NMDA Agonist Site | Formulation C | 87.73 |
| Glutamate NMDA Agonist Site | Formulation E | 71.39 |
| Glutamate NMDA Agonist Site | Formulation F | 76.81 |
| Glutamate NMDA Agonist Site | Formulation G | 94.73 |
| Glutamate NMDA Agonist Site | Formulation H | 97.52 |
| Glutamate NMDA Agonist Site | Formulation I | 38.1 |
| Glutamate Transport | Formulation A | −0.94 |
| Glutamate Transport | Formulation A | 7.92 |
| Glutamate Transport | Formulation B | −76.76 |
| Glutamate Transport | Formulation B | −24.58 |
| Glutamate Transport | Formulation C | 48.27 |
| Glutamate Transport | Formulation E | −23.39 |
| Glutamate Transport | Formulation F | 34.04 |
| Glutamate Transport | Formulation G | 19.63 |
| Glutamate Transport | Formulation H | 34.16 |
| Glutamate Transport | Formulation I | 83.47 |
| Leukotriene B4 | Formulation A | 24.54 |
| Leukotriene B4 | Formulation F | 87.58 |
| Leukotriene D4 | Formulation A | 12.49 |
| Leukotriene D4 | Formulation F | −5.47 |
| Melatonin | Formulation A | 1.92 |
| Melatonin | Formulation B | −0.19 |
| Melatonin | Formulation I | 14.99 |
| Norepinephrine Transporter | Formulation A | 58.88 |
| Norepinephrine Transporter | Formulation A | 0.68 |
| Norepinephrine Transporter | Formulation B | 29.7 |
| Norepinephrine Transporter | Formulation B | 13.29 |
| Norepinephrine Transporter | Formulation C | 32.99 |
| Norepinephrine Transporter | Formulation E | 10.9 |
| Norepinephrine Transporter | Formulation F | 27.77 |
| Norepinephrine Transporter | Formulation G | 25.83 |
| Norepinephrine Transporter | Formulation H | 21.14 |
| Norepinephrine Transporter | Formulation I | 12.66 |
| Opiate Kappal | Formulation A | 27.52 |
| Opiate Kappal | Formulation F | 50.62 |
| Peripheral Benzodiazepine | Formulation A | 79.05 |
| Peripheral Benzodiazepine | Formulation B | 7.22 |
| Peripheral Benzodiazepine | Formulation I | 69.02 |
| Platelet Activating Factor | Formulation A | 8.13 |
| Platelet Activating Factor | Formulation F | −2.55 |
| Serotonin 5-HT1 | Formulation A | 60.1 |
| Serotonin 5-HT1 | Formulation B | 25.86 |
| Serotonin 5-HT1 | Formulation C | 9.88 |
| Serotonin 5-HT1 | Formulation E | −26.66 |
| Serotonin 5-HT1 | Formulation F | 23.12 |
| Serotonin 5-HT1 | Formulation G | −19.45 |
| Serotonin 5-HT1 | Formulation H | −1.66 |
| Serotonin Non-selective | Formulation A | 29.24 |
| Serotonin Non-selective | Formulation A | 0.81 |
| Serotonin Non-selective | Formulation B | 13.48 |
| Serotonin Non-selective | Formulation B | 26.87 |
| Serotonin Non-selective | Formulation C | 21.2 |
| Serotonin Non-selective | Formulation E | 0.14 |
| Serotonin Non-selective | Formulation F | 4.95 |
| Serotonin Non-selective | Formulation G | 30.48 |
| Serotonin Non-selective | Formulation H | 36.13 |
| Serotonin Non-selective | Formulation I | 16.42 |
| Serotonin Transporter | Formulation A | 93.94 |
| Serotonin Transporter | Formulation A | −3.54 |
| Serotonin Transporter | Formulation B | 90.08 |
| Serotonin Transporter | Formulation B | 96.31 |
| Serotonin Transporter | Formulation C | 8.95 |
| Serotonin Transporter | Formulation E | 39.25 |
| Serotonin Transporter | Formulation F | 62.4 |
| Serotonin Transporter | Formulation G | −4.01 |
| Serotonin Transporter | Formulation H | −8.4 |
| Serotonin Transporter | Formulation I | 87.02 |
| Thromboxane A2 | Formulation A | 29.89 |
| Thromboxane A2 | Formulation F | 44.71 |

What is claimed is:

1. A composition comprising a plant extract substantially free of obacunone and limonin wherein the plant belongs to the family Rutaceae and the composition is suitable for ingestion by a mammal.

2. An aqueous Rutaceae plant extract substantially free of obacunone and limonin.

3. A Rutaceae plant extract comprising at least two compounds selected from the group consisting of phellodendrine, magnoflorine, and berberine wherein the Rutaceae plant was extracted using a solvent selected from the group consisting of a lower alcohol, water, or mixtures thereof.

4. A composition comprising the Rutaceae plant extract of claim 1, 2, or 3 and a pharmaceutically acceptable carrier or excipient.

5. The composition according to claim 4, wherein the composition is a pharmaceutical composition for human use.

6. A unit dosage form comprising the Rutaceae plant extract of claim 1, 2, or 3 and a physiologically acceptable diluent or carrier.

7. The unit dosage form according to claim 6 suitable for parenteral or oral administration to a human.

8. The composition according to claim 1, wherein the Rutaceae plant extract is soluble in a lower alcohol, water, and mixtures thereof.

9. The composition according to claims 1, 2 or 3, wherein the Rutaceae plant belongs to the genus Phellodendron, Citrus, Evodia, or Dictamnus.

10. The composition according to claim 1, wherein the Rutaceae plant extract is present in an amount of about 25% to about 100% by weight of the composition.

11. The pharmaceutical composition according to claim 5, further comprising at least one anxiolytic agent.

12. The pharmaceutical composition according to claim 11, wherein the anxiolytic agent is selected from the group consisting of barbituates, serotonin reuptake inhibitors, benzodiazepines, monoamine oxidase inhibitors, and mixtures thereof.

13. The pharmaceutical composition according to claim 12, wherein the anxiolytic agent is selected from the group consisting of Kava Kava, St. John's Wart, hydroxyzine hydrochloride, buspirone, mephobarbital, meprobamate, paroxetine, perphenazine, amitriptyline, fluvoxamine, sertraline, fluoxetine, alprazolam, chlordiazepoxide, clonazepam, clorazepate, diazepam, lorazepam, oxazepam, phenelzine, tranylcypromine, prazepam, halazepam, azapirones, gepirone, ipsopirone, tiaspirone, clozapine, fluperlapine, olanzapine, zotapine, seroquel, benzepine, preclamol, pramipexole, emonapride, eticlopride, raclopride, remoxipride, hydroxyaminotetralins, hexahydrobenzophenanthridines, and mixtures thereof.

14. The pharmaceutical composition according to claim 5, further comprising at least one anti-depressant agent.

15. The pharmaceutical composition according to claim 14, wherein the anti-depressant agent is selected from the group consisting of tricyclic antidepressants, monoamine oxidase inhibitors, and serotonin reuptake inhibitors.

16. The pharmaceutical composition according to claim 15, wherein the anti-depressant agent is selected from the group consisting of mirtazapine, nefazodone, bupropion, amitriptyline, amoxapine, clomipramine, desipramine, doxepin, imipramine, maprotiline, nortriptyline, protriptyline, trazodone, trimipramine, venlafaxine, paroxetine, perphenazine, amitriptyline, fluvoxamine, sertraline, fluoxetine, isocarboxazid, pargyline, phenelzine, tranylcypromine, dibenzazepines, selegiline, and mixtures thereof.

17. The pharmaceutical composition according to claim 5, further comprising at least one analgesic agent.

18. The pharmaceutical composition according to claim 17, wherein the analgesic is selected from the group consisting of non-sedating agents, dietary aids that have efficacy against pain and chronic pain, and nonsteroidal anti-inflammatory drugs.

19. The pharmaceutical composition according to claim 18, wherein the analgesic is selected from the group consisting of aspirin, ibuprofen, ketoprofen, naproxen, acetaminophen, choline magnesium trisalicylate, diclofenac, diflunisal, fenoprofen, flurbiprofen, indomethacin, meclofenamate, nabumetone, oxaprozin, phenylbutazone, piroxicam, salsalate, sulindac, tolmetin, sodium salicylate, salicylsalicylic acid, sulfasalazine, olsalazine, etodolac, keterolac, mefenamic acid, meclofenamic acid, oxicams, piroxicam, tenoxicam, pyrazolidinediones, phenylbutazone, oxyphenthatrazone, and mixtures thereof.

20. A Rutaceae plant extract obtained by the process of:
cutting or pulverizing a plant of the family Rutaceae;
extracting the cut or powdered plant parts with a suitable aqueous solvent at refluxing temperature for a time sufficient to form an extract; and
concentrating the extract under reduce pressure to obtain a Rutaceae plant extract.

21. A Rutaceae plant extract comprising at least two compounds selected from the group consisting of phellodendrine, magnoflorine, and berberine obtained by extracting a plant of the family Rutaceae with an aqueous organic solution, separating and isolating the solution and reducing solution in volume.

22. A method for treating or preventing anxiety in a mammal which comprises administering to a mammal in need thereof a therapeutically effective amount of Rutaceae plant or a composition thereof wherein the Rutaceae plant belongs to the genus Phellodendron, Citrus, Evodia, or Dictamnus.

23. A method for treating or preventing anxiety in a mammal which comprises administering to a mammal in need of anxiety treatment a therapeutically effective amount of an aqueous Rutaceae plant extract or a composition thereof.

24. The method according to claim 23, wherein the extract is obtained using a solvent selected from the group consisting of a lower alcohol, water, and mixtures thereof.

25. The method according to claim 23, wherein the Rutaceae plant extract is substantially free of obacunone or limonin.

26. The method according to claim 24, wherein the Rutaceae plant extract comprises at least two compounds selected from the group consisting of phellodendrine, magnoflorine, and berberine.

27. A method for treating or preventing depression in a mammal which comprises administering to a mammal in need thereof a therapeutically effective amount of a Rutaceae plant or a composition thereof wherein the Rutaceae plant belongs to the genus Phellodendron, Citrus, Evodia, or Dictamnus.

28. A method for treating or preventing depression in a mammal which comprises administering to a mammal in need thereof a therapeutically effective amount of an aqueous Rutaceae plant extract or a composition thereof.

29. The method according to claim 28, wherein the extract is obtained using a solvent selected from the group consisting of a lower alcohol, water, and mixtures thereof.

30. The method according to claim 28, wherein the Rutaceae plant extract is substantially free of obacunone or limonin.

31. The method according to claim 29, wherein the Rutaceae plant extract comprises at least two compounds from selected from the group consisting of phellodendrine, magnoflorine, and berberine.

32. A method for treating or preventing pain in a mammal which comprises administering to a mammal in need thereof a therapeutically effective amount of a Rutaceae plant or a composition thereof wherein the Rutaceae plant belongs to the genus Phellodendron, Citrus, Evodia, or Dictamnus.

33. A method for treating or preventing pain in a mammal which comprises administering to a mammal in need of pain treatment a therapeutically effective amount of an aqueous Rutaceae plant extract or a composition.

34. The method according to claim 33, wherein the extract is obtained using a solvent selected from the group consisting of a lower alcohol, water, and mixtures thereof.

35. The method according to claim 33, wherein the Rutaceae plant extract is substantially free of obacunone or limonin.

36. The method according to claim 34, wherein the Rutaceae plant extract comprises at least two compounds from selected from the group consisting of phellodendrine, magnoflorine, and berberine.

37. A method for treating or preventing chronic pain in a mammal which comprises administering to a mammal in need thereof a therapeutically effective amount of a Rutaceae plant or a composition thereof wherein the Rutaceae plant belongs to the genus Phellodendron, Citrus, Evodia, or Dictamnus.

38. A method for treating or preventing chronic pain in a mammal which comprises administering to a mammal in need thereof a therapeutically effective amount of an aqueous Rutaceae plant extract or a composition thereof.

39. The method according to claim 38, wherein the extract is obtained using a solvent selected from the group consisting of a lower alcohol, water, and mixtures thereof.

40. The method according to claim 38, wherein the Rutaceae plant extract is substantially free of obacunone or limonin.

41. The method according to claim 39, wherein the Rutaceae plant extract comprises at least two compounds from selected from the group consisting of phellodendrine, magnoflorine, and berberine.

42. A method for treating or preventing premenstrual syndrome in a mammal which comprises administering to a mammal in need thereof a therapeutically effective amount of a Rutaceae plant or a composition thereof wherein the Rutaceae plant belongs to the genus Phellodendron, Citrus, Evodia, or Dictamnus.

43. A method for treating or preventing premenstrual syndrome in a mammal which comprises administering to a mammal in need thereof a therapeutically effective amount of an aqueous Rutaceae plant extract or a composition thereof.

44. The method according to claim 43, wherein the extract is obtained using a solvent selected from the group consisting of a lower alcohol, water, and mixtures thereof.

45. The method according to claim 43, wherein the Rutaceae plant extract is substantially free of obacunone or limonin.

46. The method according to claim 44, wherein the Rutaceae plant extract comprises at least two compounds from selected from the group consisting of phellodendrine, magnoflorine, and berberine.

47. A method for treating or preventing separation anxiety for domestic animals which comprises administering to a mammal in need thereof a therapeutically effective amount of a Rutaceae plant extract or a composition thereof wherein the Rutaceae plant belongs to the genus Phellodendron, Citrus, Evodia, or Dictamnus.

48. The method according to claim 47, wherein the extract is obtained using a solvent selected from the group consisting of a lower alcohol, water, and mixtures thereof.

49. The method according to claim 47, wherein the Rutaceae plant extract is substantially free of obacunone or limonin.

50. The method according to claim 48, wherein the Rutaceae plant extract comprises at least two compounds from selected from the group consisting of phellodendrine, magnoflorine, and berberine.

51. A method for treating or preventing a disorder selected from the group consisting of weight loss, cold, appetite loss, sleeplessness, and fatigue which comprises administering to a mammal in need thereof a therapeutically effective amount of a Rutaceae plant extract or a compositions thereof wherein the Rutaceae plant belongs to the genus Phellodendron, Citrus, Evodia, or Dictamnus.

52. A method for treating or preventing a disorder selected from the group consisting of anxiety, depression, and premenstrual syndrome which comprises administering to a mammal in need thereof a therapeutically effective amount of berberine, a pharmaceutically acceptable salt thereof, or a composition thereof.

53. A method for treating or preventing a disorder selected from the group consisting of weight loss, cold, appetite loss, sleeplessness, and fatigue which comprises administering to a mammal in need thereof a therapeutically effective amount of berberine, a pharmaceutically acceptable salt thereof, or a composition thereof.

54. A method for treating or preventing a disorder selected from the group consisting of anxiety, pain, chronic pain, depression, and premenstrual syndrome which comprises administering to a mammal in need thereof a therapeutically effective amount of phellodendrine or magnoflorine, a pharmaceutically acceptable salt thereof, or a composition thereof.
